(19) United States
(12) Patent Application Publication
(10) Pub. No.: US 2010/0064411 A1
(43) Pub. Date: Mar. 18, 2010

(54) ARM-SECURING GARMENT

(76) Inventor: Anita Price, Massillon, OH (US)

Correspondence Address: KRUGLIAK, WILKINS, GRIFFITHS & DOUGHERTY CO, LPA, 4775 MUNSON STREET N.W., P.O. BOX 36963, CANTON, OH, 44735-6963, US

(21) Appl. No.: 12/556,615

(22) Filed: Sep. 10, 2009

Related U.S. Application Data
(60) Provisional application No. 61/096,400, filed on Sep. 12, 2008.

(51) Int. Cl. A41D 1/00; A61F 5/00; A41D 27/00

(52) U.S. Cl. 2/69; 602/4; 2/243.1

(57) ABSTRACT

An arm-securing garment for immobilizing an arm of a user to promote healing of an injured collarbone and/or arm, while being comfortable for the user, is provided. The garment includes a back panel, a right side panel attached to and extending from a right side of the back panel, and a left side panel attached to and extending from a left side of the back panel. A sleeve is affixed to and extends generally across an interior of at least one of the right side panel and the left side panel. Fastening means secures the right side panel and the left side panel to one another, so that when the right side panel and the left side panel are secured to one another, the interior sleeve is capable of holding the arm of the user in a generally uniform position against a front of the user's torso.

### CROSS-REFERENCE TO RELATED APPLICATIONS

This application claims the benefit of U.S. Provisional Patent Application Ser. No. 61/096,400, which was filed on Sep. 12, 2008.

### BACKGROUND OF THE INVENTION

1. Technical Field

The present invention relates to the art of garments. More particularly, the invention relates to the art of garments that are capable of securing a person's arm to promote the healing of a collarbone or arm injury. Still more particularly, the present invention is directed to a garment with an integrated interior sleeve that secures and generally immobilizes the arm of a user, which promotes the healing of an injured collarbone or arm with minimal inconvenience for the user and/or a person caring for the user.

2. Background Art

Reference herein will at times be made to the term child or children as a user of the present invention. It is to be understood that such reference is by way of example for the purpose of convenience, and that users of the invention include newborn babies, infants, and toddlers, as well as older individuals, such as geriatric patients, disabled persons, and other people who have suffered collarbone and/or arm injuries and need to have an arm immobilized for healing.

Collarbone injuries are one of the leading injuries experienced by children. When a collarbone injury is experienced by a child, there is a need to immobilize the arm of the child that is adjacent the injured collarbone, so that substantial movement of the arm is prevented. This immobilization enables the injured collarbone to heal. Such immobilization is also desirable when a child experiences a shoulder, elbow or other arm injury, as the prevention of substantial movement of the injured arm promotes healing of the injury.

For certain adults and older juveniles, immobilization of the injured arm or the arm adjacent the injured collarbone is typically performed by securing the arm to the person's torso by a sling. However, for younger children or other individuals, a sling is often difficult and/or impractical to employ, since slings are often uncomfortable for the user and such users typically do not realize the necessity and/or structure of the sling. For example, a child may resist the installation of a sling by an adult who is caring for the child, thus making it difficult to put on, and/or the child may become undesirably tangled in the sling. Despite such difficulty, it remains desirable to attempt to immobilize the user's arm in order to promote the healing of an injured collarbone or an injured arm.

As a result, there is a need in the art for an apparatus that is able to immobilize an arm of a user, which is easy to put on the user, is simple and safe to use, and is comfortable for the user. The arm-securing garment of the present invention satisfies this need.

### BRIEF SUMMARY OF THE INVENTION

An objective of the present invention is to provide an apparatus that is capable of generally immobilizing the arm of a user.

Another objective of the present invention is to provide an apparatus that is easy to put on a user, and is relatively simple, safe and comfortable to use.

These objectives and others are obtained by the arm-securing garment of the present invention. In an exemplary embodiment of the invention, the arm-securing garment immobilizes an arm of a user to promote healing of an injured collarbone and/or arm. The garment includes a back panel, a right side panel attached to and extending from a right side of the back panel, and a left side panel attached to and extending from a left side of the back panel. A sleeve is affixed to and extends generally across an interior of at least one of the right side panel and the left side panel. Fastening means secures the right side panel and the left side panel to one another, so that when the right side panel and the left side panel are secured to one another, the interior sleeve is capable of holding the arm of the user in a generally uniform position against a front of the user's torso.

### BRIEF DESCRIPTION OF THE SEVERAL VIEWS OF THE DRAWINGS

The preferred embodiment of the present invention, illustrative of the best mode in which applicant has contemplated applying the principles, is set forth in the following description and is shown in the drawings, and is particularly and distinctly pointed out and set forth in the appended claims.

FIG. 1 is a Font elevational view of an exemplary embodiment of an arm-securing garment of the present invention, shown in a closed, or fastened, position; and

FIG. 2 is a front elevational view of the arm-securing garment shown in FIG. 1, shown in an open position.

Similar numerals refer to similar parts throughout the drawings.

### DETAILED DESCRIPTION OF THE INVENTION

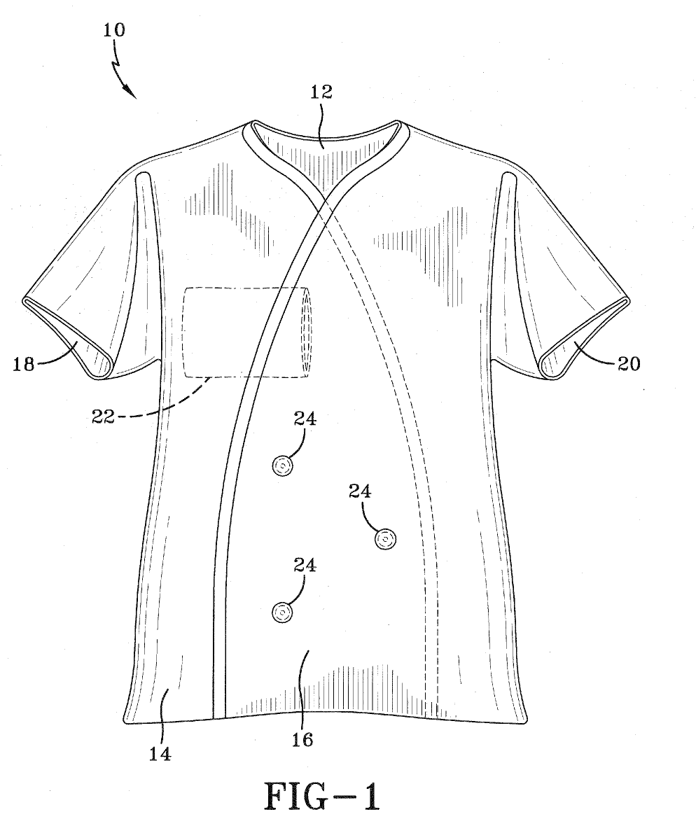

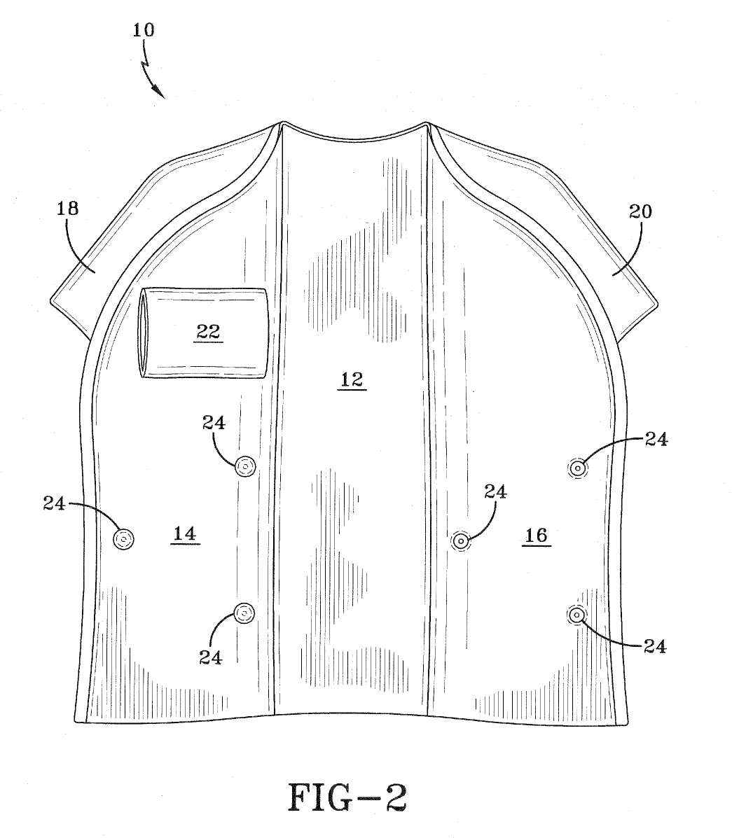

Turning now to the drawings of the present invention, wherein the illustrations are for showing the preferred embodiment of the invention, and not for limiting the same, FIGS. 1-2 show an exemplary embodiment of an arm-securing garment of the present invention, indicated generally at 10. Garment 10 generally surrounds the user's torso (not shown), and generally is worn in the manner of a shirt.

Garment 10 includes a back panel 12, which covers the back of the user's torso. Reference herein shall be made to right side and left side for the purpose of convenience, with the understanding that reference to right side means the wearer's or user's right side, and reference to left side means the wearer's or user's left side. Attached to and extending from the right side of back panel 12 is a right side panel 14, which extends about the right side of the user's torso and generally across the front of the user's torso, as will be described in greater detail below. Attached to and extending from the left side of back panel 12 is a left side panel 16, which extends about the left side of the user's torso and generally across the front of the user's torso, as will be described in greater detail below.

A right side exterior sleeve 18 is connected to and extends from right side panel 14, so that if the user's right collarbone and right arm are uninjured, the user's right arm extends through the right side exterior sleeve. A left side exterior sleeve 20 is connected to and extends from left side panel 16, so that if the user's left collarbone and left arm are uninjured, the user's left arm extends through the left side exterior sleeve.

Connected to and extending generally across the interior of one of right side panel 14 and left side panel 16 is an interior, generally tubular-shaped, sleeve 22. In FIGS. 1 and 2, interior sleeve 22 is shown connected or affixed to right side panel 14, in which case the injured collarbone or arm is on the user's right side. It is to be understood that interior sleeve 22 is shown connected to right side panel 14 by way of example, as the interior sleeve may alternatively be connected or affixed to left side panel 16 if the injured collarbone or arm is on the left side of the user. Alternatively, two interior sleeves 22 may by employed, with one connected or affixed to right side panel 14 and one connected or affixed to left side panel 16, so that garment 10 may easily accommodate an injury to a collarbone or arm on either one or both of the user's right and left sides.

Interior sleeve 22 is connected or affixed to the interior of right side panel 14 by stitching or other means. For example, permanent means, such as heavy stitching, mechanical fasteners, adhesives, or integral formation may be used to connect interior sleeve 22 to the right side panel 14. Alternatively, temporary means, such as light stitching that can readily be cut when desired, Velcro fasteners, snaps, ties, buttons, hooks, or loops may be used to connect interior sleeve 22 to the right side panel 14. In its open position (FIG. 2), garment 10 preferably is an open-style garment, such as a shirt, onesie, or sleeper-style garment, which enables the garment to be easily put on the user.

More particularly, to put garment 10 on the user, the person caring for or assisting the user slides interior sleeve 22 over the forearm of the user. Once interior sleeve 22 securely captures and surrounds the user's forearm and/or elbow, back panel 12 is pulled around the back of the user's torso, and left panel 16 is pulled around the user's left side to enable the user's uninjured left arm to be inserted through left side exterior sleeve 22. In this manner, garment 10 is securely wrapped around the torso of the user.

Once garment 10 is wrapped around the user's torso, snaps 24 or other fastening means are used to secure left side panel 16 to right side panel 14 on the front of the user's torso. For example, three snaps 24 may be used to secure left side panel 16 to right side panel 14. It is to be understood that such fastening means may include snaps, buttons, ties, hooks, loops, Velcro fasteners, and other closure means, as known in the art. In addition, the number and location of snaps 24 and/or other fastening means may be adapted to suit particular aesthetic and/or closure requirements of garment 10.

Preferably, garment 10 is formed of cotton or other suitable material, such as nylon, polyethylene, and/or other natural or synthetic fabrics that are known to those skilled in the art, including combinations thereof. Selected components of garment 10, such as interior sleeve 22, may be of different materials than the remainder of the garment. For example, interior sleeve 22 may be made of a resilient synthetic fabric, such as neoprene or other material, in order to securely support the user's arm, while the remainder of garment 10 may be made of cotton for comfort and durability.

The vertical and horizontal position of interior sleeve 22 on right side panel 14 is set to provide a suitable location to secure the user's forearm of the user against the middle area of the user's torso. Preferably, interior sleeve 22 extends generally horizontally across right side panel 14 to secure the user's forearm in a generally horizontal position. In addition, the preferred vertical position or location of interior sleeve 22 is generally below right side exterior sleeve 18 and above the vertical mid-point of right side panel 14. Of course, other positions of interior sleeve 22 may be employed in order to secure the user's arm in a different position or orientation, as may be needed. For example, interior sleeve 22 may be disposed in a vertically higher or lower position along right side panel 14, or at an angled orientation relative to horizontal.

In addition, the diameter and length of generally tubular-shaped interior sleeve 22 is suitably designed to slide over the user's hand and capture and support the user's forearm, and may optionally capture and support the user's elbow as well. For example, when garment 10 is intended for use by an infant, interior sleeve 22 is formed with a diameter and length suitable to capture the forearm, and optionally the elbow, of a typical infant. Likewise, when garment 10 is intended for use by an adult, such as a geriatric patient, interior sleeve 22 is formed with a diameter and length suitable to capture the forearm, and optionally the elbow, of a typical geriatric patient.

Once left side panel 16 is fastened to right side panel 14, which closes garment 10, interior sleeve 22 holds the user's arm in a generally uniform and immobile position against the front of his or her torso. This immobilization promotes healing of the injured collarbone and/or arm, while being comfortable for the user.

Arm-securing garment 10 of the present invention thus provides a garment that conveniently and easily immobilizes a user's arm in order to promote the healing of an injured collarbone and/or arm. Interior sleeve 22 is integrated into garment 10, and is easily pulled over the forearm of the arm that is to be immobilized, while the remainder of the garment is easily wrapped around the user. Once garment 10 is fastened and thus closed, interior sleeve 22 securely holds the user's arm in a desired position. Interior sleeve 22 is more comfortable, safer, and more secure than a prior art sling, and enables garment 10 to be easily put on the user by a person assisting the user. Moreover, the use of interior sleeve 22 as an integral part of garment 10 provides an economical garment that may be used after healing of the injured area is complete.

It is to be understood that interior sleeve 22 of garment 10 may be located on either right side panel 14 or left side panel 16. In addition, an interior sleeve 22 may be transferable between each of right side panel 14 and left side panel 16, or an interior sleeve may be employed on each one of the right side panel and the left side panel, so that garment 10 may easily accommodate an injury to a collarbone or arm on either one of the user's right and left sides, without affecting the overall concept or operation of the invention. Moreover, as mentioned above, interior sleeve 22 may be permanently affixed to garment 10, or temporarily affixed to the garment, without affecting the overall concept or operation of the invention. While interior sleeve 22 has been described as being generally tubular-shaped, it may be configured in other shapes to suit particular design requirements.

Reference above has been made to the term child or children as a user of the present invention. It is to be understood that such reference has been by way of example for the purpose of convenience, and that users of the invention include newborn babies, infants, and toddlers, as well as older individuals, such as geriatric patients, disabled persons, and other people who have suffered collarbone and/or arm injuries and need to have an arm immobilized for healing.

The present invention also includes a method for immobilizing an arm using an arm-securing garment. The method generally includes the steps of providing an arm-securing garment and installing the garment on a user to secure or hold the user's arm in a generally uniform and immobile position against the front of the user's torso to promote healing of an injured collarbone and/or arm, and includes steps in accordance with the description that is presented above and shown in FIGS. 1-2.

Accordingly, the improved arm-securing garment is simplified, provides an effective, safe, inexpensive, and efficient structure which achieves all the enumerated objectives, provides for eliminating difficulties encountered with prior art arm-securing devices, and solves problems and obtains new results in the art.

In the foregoing description, certain terms have been used for brevity, clarity and understanding; but no unnecessary limitations are to be implied therefrom beyond the requirements of the prior art, because such terms are used for descriptive purposes and are intended to be broadly construed. Moreover, the present invention has been described with reference to exemplary embodiments. It shall be understood that this illustration is by way of example and not by way of limitation, as the scope of the invention is not limited to the exact details shown or described. Potential modifications and alterations will occur to others upon a reading and understanding of this disclosure, and it is understood that the invention includes all such modifications and alterations and equivalents thereof.

Having now described the features, discoveries and principles of the invention, the manner in which the improved arm-securing garment is constructed, arranged and used, the characteristics of the construction and arrangement, and the advantageous, new and useful results obtained; the new and useful structures, devices, elements, arrangements, parts and combinations are set forth in the appended claims.

What is claimed is:

1. An arm-securing garment, comprising:
a back panel;
a right side panel attached to and extending from a right side of said back panel;
a left side panel attached to and extending from a left side of said back panel;
a sleeve affixed to and extending generally across an interior of at least one of said right side panel and said left side panel; and
fastening means to secure said right side panel and said left side panel to one another, whereby when the right side panel and the left side panel are secured to one another, said interior sleeve is capable of holding an arm of a user of said garment in a generally uniform position against a front of said user's torso.

2. The arm-securing garment of claim 1, wherein said sleeve is formed with a tubular shape.

3. The arm-securing garment of claim 1, wherein said fastening means includes at least one of snaps, buttons, ties, hooks, loops, and Velcro fasteners.

4. The arm-securing garment of claim 1, wherein said sleeve extends generally horizontally across an interior of at least one of said right side panel and said left side panel.

5. The arm-securing garment of claim 1, wherein said garment further comprises a right side exterior sleeve attached to and extending from said right side panel, and said interior sleeve is affixed to the right side panel generally below said right side exterior sleeve and a vertical mid-point of said right side panel.

6. The arm-securing garment of claim 1, wherein said garment further comprises a left side exterior sleeve attached to and extending from said left side panel, and said interior sleeve is affixed to the left side panel generally below said left side exterior sleeve and a vertical mid-point of said left side panel.

7. The arm-securing garment of claim 1, wherein said sleeve is affixed to said interior of at least one of said right side panel and said left side panel by at least one of heavy stitching, mechanical fasteners, adhesives, and integral formation.

8. The arm-securing garment of claim 1, wherein said sleeve is affixed to said interior of at least one of said right side panel and said left side panel by at least one of light stitching, Velcro fasteners, snaps, ties, buttons, hooks, and loops.

9. The arm-securing garment of claim 1, wherein said garment includes at least one of a shirt, a onesie, and a sleeper-style garment.

10. The arm-securing garment of claim 1, further comprising two sleeves, wherein a first one of said sleeves is affixed to and extends generally across an interior of said right side panel, and second one of said sleeves is affixed to and extends generally across an interior of said left side panel.

11. A method for immobilizing an arm using said arm-securing garment of claim 1.
